(19) United States
(12) Patent Application Publication
(10) Pub. No.: US 2018/0200411 A1
(43) Pub. Date: Jul. 19, 2018

(54) MATRIX CONSTRUCTION

(71) Applicant: DermaGenesis, LLC, Pompano Beach, FL (US)

(72) Inventors: Jess Bills, Pompano Beach, FL (US); Derek Cox, Pompano Beach, FL (US); David Tumey, Pompano Beach, FL (US)

(21) Appl. No.: 15/907,417

(22) Filed: Feb. 28, 2018

Related U.S. Application Data
(62) Division of application No. 15/631,530, filed on Jun. 23, 2017.
(60) Provisional application No. 62/353,620, filed on Jun. 23, 2016.

(51) Int. Cl. A61L 27/60; A61L 27/56; C08H 1/00; A61L 27/26; F26B 5/06

(52) U.S. Cl. CPC A61L 27/60; A61L 27/56; C08H 1/00; A61L 2430/34; A61L 2430/02; F26B 5/06; A61L 27/26

(57) ABSTRACT

For making a matrix useable in a wound, matrix construction methods are provided, in which a slurry is formulated and then lyophilized. Usage of collagen and chondroitin sulfate (C6S) in the slurry is favored.

### FIELD OF THE INVENTION

The invention relates to technology in support of tissue grafting, and more particularly, skin grafting.

### BACKGROUND OF THE INVENTION

Recently, new technology has been invented in which customized skin grafts are produced from harvested living cells. See US 20150140058 published May 21, 2015; US 20150139960 published May 21, 2015; US 20150366655 published Dec. 24, 2015. An aspect of making skin grafts from harvested cells has been to print (via a 3-D printer) harvested cells onto a pre-constructed base or “matrix”. For example, a substrate can be constructed by 3-D-printing sheets of a biosorbable material integrated with a collagen matrix. In one approach, prefab same-size sheets of collagen matrix with or without a honeycomb substrate can be trimmed to a shape of a wound. Custom-printing a collagen matrix would be another approach, but can add further time and complexity.

### SUMMARY OF THE INVENTION

An objective of the invention is to provide methods of producing matrices useable in the production of tissue grafts made from harvested living cells. The invention is especially directed to producing matrices useable in production of skin grafts; matrices useable in production of other tissue grafts, such as bone grafts, etc., also are within the scope of the invention.

A further objective of the invention is to provide methods of producing an acellular matrix component of a graft that will be placed into a patient's wound.

The invention in one preferred embodiment provides a method of making a matrix useable in a wound, comprising the steps of: a) formulating a slurry from a set of solid components comprising collagen and chondroitin sulfate (C6S); and b) performing a lyophilization step whereby aqueous components are removed.

In another preferred embodiment, the invention provides a method of making a matrix useable in a wound, comprising the steps of: a) containing a slurry (such as, e.g., a slurry that comprises collagen and chondroitin sulfate; a slurry that comprises collagen, chondroitin sulfate and Hyaluronic Acid (HA); a slurry that comprises collagen, chondroitin sulfate, HA and fibronectin; a slurry that comprises acetic acid; a slurry that comprises elastin (ELN) alone or optionally with at least one selected from the group consisting of C6S, HA and FN; a slurry that comprises Tenascin (TEN) alone or optionally with at least one selected from the group consisting of C6S, HA and FN) in a matrix carrier, which is not a tray, wherein the matrix carrier comprises a wound-shaped cavity having a size and shape duplicative of a wound; and b) performing a lyophilization step on the slurry while contained in the matrix carrier.

The invention in a preferred embodiment provides a method of making a matrix useable in a wound, comprising the steps of: formulating a slurry from a set of solid components comprising collagen and chondroitin sulfate (C6S) (such as, e.g., formulating collagen, C6S and hyaluronic acid (HA) into a slurry; formulating collagen, C6S, HA and fibronectin (FN) into a slurry; formulating collagen and ELN into a slurry; formulating collagen, C6S and ELN into a slurry; formulating collagen, C6S, ELN and HA into a slurry; formulating collagen, C6S, ELN, HA and FN into a slurry; formulating collagen and TEN into a slurry; formulating collagen, C6S and TEN into a slurry; formulating collagen, C6S, TEN and HA into a slurry; formulating collagen, C6S, TEN, HA and FN into a slurry; formulating collagen, C6S, TEN, HA, FN and ELN into a slurry; formulating collagen and one or more of C6S, TEN, HA, FN and/or ELN into a slurry; formulating collagen, chondroitin sulfate (C6S) and FN into a slurry; and other slurry--formulating steps); and performing a lyophilization step whereby aqueous components are removed (such as, e.g., a lyophilization step performed when the slurry is contained in a matrix carrier comprising a wound-shaped cavity having a size and shape duplicative of a wound), such as, e.g., inventive methods comprising performing a cross-linking step after the lyophilization step; inventive methods wherein in the slurry-formulating step, the slurry has a solids fraction that consists of collagen and C6S; inventive methods wherein in the slurry-formulating step, the slurry has a solids fraction that consists of collagen, C6S, and HA; inventive methods wherein in the slurry-formulating step, the slurry has a solids fraction that consists of collagen, C6S, HA and fibronectin; inventive methods wherein the slurry-formulating step comprises adding collagen to a solution, followed by adding fibronectin to the solution, followed by adding chondroitin sulfate to the solution, followed by adding hyaluronic acid to the solution; inventive methods comprising, after collagen-adding and before fibronectin-adding, blending for a period of time until chunks are completely dissolved; inventive methods wherein the slurry-formulating comprises adding solid components to acetic acid (such as, e.g., adding solid components to acetic acid at a concentration in a range of from 0.01 to 1.0 molarity; adding solid components to acetic acid at a concentration of 0.05 molarity; adding solid components to acetic acid at a concentration of 0.001-10 M of the acid; steps of mixing 0.286 mL of glacial acetic acid with 99.714 mL of distilled water to obtain 100 mL of 0.05 molar acetic acid solution, followed by adding solid components into the 0.05 molar acetic acid solution; etc.); inventive methods comprising adding collagen into a solution to produce a final solution in a range of about 0.5 to 1.0%; inventive methods comprising adding collagen into a solution to produce a 0.5% final solution; inventive methods comprising adding C6S into a solution to produce a final solution in a range of about 0.001-25%; inventive methods comprising adding C6S into a solution to produce a 0.02% final solution; inventive methods comprising adding HA into a solution to produce a final solution in a range of about 0.001-25%; inventive methods comprising adding HA into a solution to produce a 0.02% final solution; inventive methods comprising adding fibronectin into a solution to produce a final solution in a range of about 0.001 to 10%; inventive methods comprising adding fibronectin into a solution to produce a 0.001% final solution; inventive methods comprising adding fibronectin into a solution in which the range for the ELN and/or TEN is 0.001-10%; inventive methods comprising mixing at least one vitamin into the slurry; inventive methods omprising mixing into the slurry at least one GAG selected from the group consisting of: HA, FN, chitosan; heparin sulfate; keratin sulfate; dermatan sulfate; and heparin; inventive methods wherein the slurry-formulating step proceeds for a time in a range of about 30-120 minutes, at a temperature in a range of about 0° C. to 10° C.; inventive methods wherein the lyophilizing step proceeds for a time in a range of about 24-72 hours, at a temperature in a range of about 0° C. to −80° C.; inventive methods wherein in the slurry-formulating step, a ratio of collagen to C6S is in a range of about 0.5% to 0.01%; inventive methods wherein after the aqueous components have been removed by the lyophilization step, a ratio of collagen to C6S is in a range of about 2% to 98%; inventive methods wherein the lyophilization step is performed using a tray selected from the group consisting of a stainless steel tray, a stainless steel tray comprising an anodized coating, an aluminum tray, an aluminum tray comprising an anodized coating, and a tray comprising an anodized coating; inventive methods wherein in the lyophilization step is used a tray comprising a chromate conversion coating; and other inventive methods.

In another preferred embodiment, the invention provides a method of making a matrix useable in a wound, comprising the steps of: containing a slurry (such as, e.g., a slurry that comprises collagen and chondroitin sulfate; a slurry that comprises collagen, chondroitin sulfate and HA; a slurry that comprises collagen, chondroitin sulfate, HA and fibronectin; a slurry that comprises acetic acid) in a matrix carrier, which is not a tray, wherein the matrix carrier comprises a wound-shaped cavity having a size and shape duplicative of a wound; and performing a lyophilization step on the slurry while contained in the matrix carrier, such as, e.g., inventive methods wherein the slurry-containing step proceeds for a time in a range of about 30-120 minutes, at a temperature in a range of about 0° C. to −80° C.; inventive methods wherein the lyophilizing step proceeds for a time in a range of about 24-72 hours, at a temperature in a range of about 0° C. to −80° C.; inventive methods wherein the lyophilization step is performed using a tray selected from the group consisting of a stainless steel tray, a stainless steel tray comprising an anodized coating, an aluminum tray, an aluminum tray comprising an anodized coating, and a tray comprising an anodized coating; inventive methods wherein in the lyophilization step is used a tray comprising a chromate conversion coating; and other inventive methods.

### BRIEF DESCRIPTION OF THE DRAWINGS

FIG. 1 depicts collagen 1, a base material useable in the invention for matrix construction.

FIG. 1A depicts an undesirable reaction that is to be avoided for collagen 1 to undergo when constructing a matrix according to the invention.

### DETAILED DESCRIPTION OF A PREFERRED EMBODIMENT OF THE INVENTION

The inventive matrix-construction comprises a slurry-formulating step in which a slurry is formulated from a set of solid components, which preferably comprise collagen and chondroitin sulfate (C6S). We consider collagen a preferred base component. Preferably acetic acid is used to dissolve the collagen. Although phosphoric acid does dissolve collagen, preferably phosphoric acid is avoided for slurry-formulation, because the phosphoric acid would eat away at the machinery used in a subsequent step. When acetic acid is used as the solvent in a slurry to dissolve the collagen, preferably the acetic acid is present in the mixes, until removed by lyophilization.

A suitable solvent is used to dissolve the materials. Our goal is to dissolve collagen and other components without damaging their secondary structures. An example of a molarity range for the solvent is, e.g., 0.001 M to 10 M, with a preferred range being 0.01 M-1.0 M, and a most preferred range being 0.01 M-0.05 M.

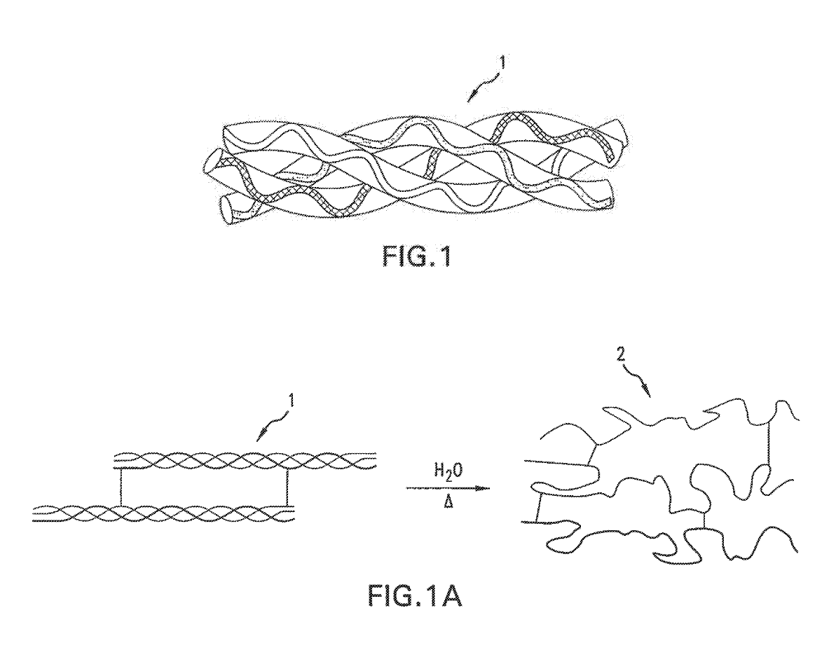

Examples of solvents for the collagen, are, e.g., acetic acid, sulfuric acid, nitric acid, phosphoric acid, hydrochloric acid, carbonic acid, formic acid, hydrofluoric acid, perchloric acid, etc. Acetic acid is most preferred as the solvent for the collagen, due to being most gentle. In addition to the above-mentioned solvents, other liquid substances also are useable to dissolve collagen. When using a liquid substance to dissolve collagen, the concentration of the solvent is chosen in order to preserve the active characteristics of collagen 1 (see FIG. 1).

A preferred construction method for matrix construction according to the invention is to use collagen 1 (FIG. 1), as follows. Collagen 1 is used as a base material in the invention, for its bioactive properties. In order for collagen 1 to remain bioactive, its triple helical secondary structure must be preserved. By failing to preserve the secondary structure of collagen 1, collagen 1 would be converted into gelatin 2 (see FIG. 1A). Gelatin 2 is no longer bioactive, and therefore conditions that bring about the conversion of collagen 1 to gelatin 2 should be avoided for constructing a matrix according to the invention.

Examples of a slurry-formulating step useable in the invention are, e.g., a slurry-formulating step that comprises formulating collagen, chondroitin sulfate (C6S) and hyaluronic acid (HA) into a slurry; a slurry-formulating step that comprises formulating collagen, chondroitin sulfate (C6S), hyaluronic acid (HA) and fibronectin (FN) into a slurry; a slurry-formulating step that comprises adding collagen to a solution, followed by adding fibronectin to the solution, followed by adding chondroitin sulfate to the solution, followed by adding hyaluronic acid to the solution (such as, e.g., a slurry-formulating step that further comprises, after the collagen-adding and before fibronectin-adding, blending for a period of time until chunks are completely dissolved); etc.

Examples of slurry-formulating are, e.g., slurry-formulating that comprises adding solid components to acetic acid (such as, e.g., slurry-formulating that comprises adding solid components to acetic acid at a concentration in a range of from 0.001 to 1.0 molarity; slurry-formulating that comprises adding solid components to acetic acid at a concentration of 0.05 molarity; etc.) In a preferred example of slurry-formulating, 0.286 mL of glacial acetic acid is mixed with 99.714 mL of distilled water to obtain 100 mL of 0.05 molar acetic acid solution, followed by adding solid components into the 0.05 molar acetic acid solution.

Examples of a slurry formed in the slurry-formulating step are, e.g., a slurry that has a solids fraction that consists of collagen and C6S; a slurry that has a solids fraction that consists of collagen, C6S, and HA; a slurry that has a solids fraction that consists of collagen, C6S, HA and fibronectin; etc.

Examples of adding collagen into a solution in a slurry-formulating step are, e.g., adding collagen into a solution to produce a final solution in a range of ˜0.1 to 10%; adding collagen into a solution to produce a final solution in a range of ˜0.2 to 5%; adding collagen into a solution to produce a final solution in a range of ˜0.5 to 2%; adding collagen into a solution to produce a final solution in a range of ˜0.5 to 1.0%; adding collagen into a solution to produce a 0.5% final solution; etc. A preferred collagen usage is in a range of 0.001 g to 100 g collagen, per 100 mL solvent (namely 0.001-10%).

Examples of adding C6S into a solution in a slurry-formulating step, are, e.g., adding C6S into a solution to produce a final solution in a range of about 0.001-10%; adding C6S into a solution to produce a 0.02% final solution; etc.

Examples of adding HA into a solution in a slurry-formulating step are, e.g., adding HA into a solution to produce a final solution in a range of about 0.02 to 0.1%; HA into a solution to produce a 0.02% final solution; etc. For adding hyaluornic acid, a preferred range is 0.001-10%.

Examples of adding fibronectin into a solution in a slurry-formulating step are, e.g., adding fibronectin into a solution to produce a final solution in a range of about 0.001 to 0.005%; adding fibronectin into a solution to produce a 0.001% final solution; etc. For adding fibronectin, a preferred range is 0.001-10%.

In the invention, some slurry-formulating steps comprise addition of elastin (such as, e.g., elastin-addition at 0.001-10%; elastin addition alone; addition of elastin with one or more selected from the group consisting of C6S, HA and FN; etc.). In the invention, some slurry-formulating steps comprise addition of Teanscin (such as, e.g., Tenascin-addition at 0.001-10%; Tenascin-addition alone; Tenascin-addition with one or more selected from the group consisting of C6S, HA and FN; etc.

Examples of adding elastin (ELN) into a solution in a slurry-formulating step are, e.g., adding ELN into a solution to produce a final solution in a range of about 0.02 to 0.1%; adding ELN into a solution to produce a 0.02% final solution; etc. For adding elastin, a preferred range is 0.001-10 g, per 100 mL solvent.

Examples of adding Tenascin (TEN) into a solution in a slurry-formulating step are, e.g., adding TEN into a solution to produce a final solution in a range of about 0.02 to 0.1%; adding TEN into a solution to produce a 0.02% final solution; etc. For adding Tenascin, a preferred range is 0.001-10 g, per 100 mL solvent.

The inventive matrix-construction comprises a lyophilization step whereby aqueous components are removed. Preferably, the lyophilization step is performed when the slurry is contained in a matrix carrier comprising a wound-shaped cavity having a size and shape duplicative of a wound. After a completed lyophilization, liquid has been completely removed as noticed by visible inspection. If lyophilization is incomplete, liquid remains as noticed by visible inspection.

Examples of a matrix carrier are, e.g., a matrix carrier that has no top cover and has a volume in a range of about 0.001 L to 50 L; a matrix carrier that has a uniform height, and a height of the matrix carrier is in a range of about 0.01 inch to 10 inches; a matrix carrier that has variable length dimensions and variable width dimensions, with a width dimension in a range of about 0.01 inch to 10 inches and a length dimension in a range of about 0.01 inch to 10 inches; etc. Various matrix carriers are useable in different embodiments. For example, a volume on an order of 50 L can be associated with a lyophilizing a system of trays. It will be appreciated that lyophilizers and trays can differ in size; in many cases, 1 L of slurry is unlikely to be enough slurry fora tray to be filled. An example of when production of large sheets might be wanted, and correspondingly use of carriers that almost completely fill a tray chamber, is in connection with a patient with severe burns. As for height of the carrier, the height dimension can vary in different embodiments. An example of a carrier with a substantial height can include carriers being made in connection with a thick appendage needing treatment. For example, the average adult male femur is 48 cm long and 2.34 cm in diameter, and a carrier to be used for producing a matrix to be used in connection with treating such a large appendage can be correspondingly sized.

After lyophilization, optionally a cross-linking step is performed.

A preferred example of a matrix carrier used in the invention is, e.g., a carrier that includes fenestration via the printer along the bottom of the carrier to allow for better uptake.

The invention may be further appreciated with reference to the following examples, without the invention being limited thereto.

### EXAMPLE 1

A matrix sponge is constructed as follows.

Primary Component. Collagen is what primarily constitutes the matrix.

Secondary Component(s). One or more secondary components, each being a glycosaminoglycan (“GAG”), is or are used to construct the matrix, along with the primary component. Examples of GAGs in this Example are Chondroitin 6-Sulfate; Hyaluronic Acid; Elastin, Tenascin and Fibronectin.

Acid Solvent. Acetic Acid (“AcAc”) is preferred, and is considered generally safer and less corrosive to machinery. Less preferred acid solvents are, e.g., ascorbic acid, hydrochloric acid, formic acid, etc. In this example, the concentration of the acid is in a range of 0.001 M-10 M.

### EXAMPLE 2 (Collagen/Acetic Acid Solution)

By addition of 0.5 g collagen to 100 mL acetic acid, a 0.5% collagen/acetic acid solution is produced.

### EXAMPLE 3 (C6S)

To a collagen/acetic acid solution, Chondroitin-6-sulphate is added in an amount of 0.020 g per 100 mL of acetic acid creating a 0.02% solution,

### EXAMPLE 4 (Slurry M1)

A slurry M1, also referred to as a dispersion, 250 mL, is produced by the following steps:

1) Make acetic acid fresh (because concentration changes after 1 week). Add 0.714 mL of glacial acetic acid to 249.286 mL of distilled water. Total 250 mL of acetic acid at 0.05 molarity, concentration.

2) Add 1.250 g collagen (bovine) to 200 mL of acetic acid at a very weak concentration (0.05 M) to achieve a 0.625% collagen solution.

3) Quick mix for about 10 seconds while moving homogenizer up and down to make sure the collagen is actually dissolving in the acetic acid.

4) Mix for 30 minutes at 24,000 rpms in homogenizer, but keep the outer blending apparatus ice cold with ice water. (The mix is to be kept cold throughout the blending process.)

5) Mix 0.050 g chondroitin 6-sulfate (C6S) to 50 mL of acetic acid (0.05 M) to achieve 0.1% C6S solution.

6) Place C6S+AcAC solution in an IV bag or peristaltic pump in which you can control the rate of the drip.

7) While homogenizing the collagen mix, begin adding C6S mix dropwise to slurry at a rate of 150 mL/hour until all 50 mL of C6S has been added. Final concentration of new solution is: 0.5% collagen and 0.02% C6S. Note: the C6S will not all be added during the first homogenizing run; the IV bag will be empty during the second cycle.

8) Depending on the homogenizer it may be very important to allow the blender to cool down after each 30 minute cycle. v9) Homogenize the collagen+C6S in acetic acid for 30 minutes at 24,000 rpms.

10) Allow homogenizer to cool down for 10 minutes if necessary.

11) Repeat steps 9 and 10, to achieve total mixing of 120 minutes minimum.

### EXAMPLE 5 (slurry M2)

A slurry M2, 250 mL, is produced by the following steps:

1) Make acetic acid fresh (because concentration changes after 1 week). Add 0.714 mL of glacial acetic acid to 249.286 mL of distilled water. Total 250 mL of acetic acid at 0.05 molarity, concentration.

2) Add 1.250 g collagen (bovine) to 200 mL of acetic acid at a very weak concentration (0.0 5M) to achieve a 0.625% collagen solution.

3) Quick mix for about 10 seconds while moving homogenizer up and down to make sure the collagen is actually dissolving in the acetic acid.

4) Mix for 30 minutes at 24,000 rpms in homogenizer, but keep the outer blending apparatus ice cold with ice water. (The mix is to be kept cold throughout the blending process.)

5) Mix 0.050 g chondroitin 6-sulfate (C6S) to 50 mL of acetic acid (0.05 M) to achieve 0.1% C6S solution.

6) Place C6S+AcAC solution in an IV bag or peristaltic pump in which you can control the rate of the drip.

7) While homogenizing the collagen mix, begin adding C6S mix dropwise to slurry at a rate of 150 mL!hour until all 50 mL of CGS has been added. Final concentration of new solution is: 0.5% collagen and 0.02% C6S. Note: the C6S will not all be added during the first homogenizing run; the IV bag will be empty during the second cycle.

8) Now add hyaluronic acid at 0.04% to the 250 mL collagen+C6S mix.

9) Quick mix for about 10 seconds while moving homogenizer up and down to make sure the HA is actually dissolving in the acetic acid.

10) Homogenize the collagen+C6S+HA in acetic acid for 30 minutes at 24,000 rpms.

11) Allow homogenizer to cool down for 10 minutes, if necessary.

12) Repeat steps 10 and 11, to achieve total mixing of 120 minutes minimum.

### EXAMPLE 6 (Slurry M3)

A slurry M3, 250 mL, is produced by the following steps:

1) Make acetic acid fresh (because concentration changes after 1 week). Add 0.714 mL of glacial acetic acid to 249.286 mL of distilled water. Total 250 mL of acetic acid at 0.05 molarity, concentration.

2) Add 1.250 g collagen (bovine) to 200 mL of acetic acid at a very weak concentration (0.05 M) to achieve a 0.625% collagen solution.

3) Quick mix for about 10 seconds while moving homogenizer up and down to make sure the collagen is actually dissolving in the acetic acid.

4) Mix for 30 minutes at 24,000 rpms in homogenizer, but keep the outer blending apparatus ice cold with ice water. (The mix is to be kept cold throughout the blending process.)

5) Mix 0.050 g chondroitin 6-sulfate (C6S) to 50 mL of acetic acid (0.05 M) to achieve 0.1% C6S solution.

6) Place C6S+AcAC solution in an IV bag or peristaltic pump in which you can control the rate of the drip.

7) While homogenizing the collagen mix, begin adding C6S mix dropwise to slurry at a rate of 150 mL/hour until all 50 mL of C6S has been added. Final concentration of new solution is: 0.5% collagen and 0.02% C6S. Note: the C6S will not all be added during the first homogenizing run; the IV bag will be empty during the second cycle.

8) Now add hyaluronic acid at 0.04% to the 250 mL collagen+C6S mix.

9) Quick mix for about 10 seconds while moving homogenizer up and down to make sure the HA is actually dissolving in the acetic acid.

10) Add the fibronectin at 0.0025 g to create a final concentration of 0.0001%.

11) Quick mix for about 10 seconds while moving homogenizer up and down to make sure the fibronectin is actually dissolving in the acetic acid.

12) Blend the collagen+C6S+HA+Fibronectin in acetic acid for 30 minutes at 24,000 rpms.

13) Allow homogenizer to cool down for 10 minutes, if necessary.

14) Repeat steps 12 and 13, to achieve total mixing of 120 minutes minimum.

### EXAMPLE 7 (Matrix Production)

In this example, a slurry (such as a slurry M1, M2 or M3 from the above examples) takes the shape of a carrier. A thickness desired for the matrix is selected. We have been making matrices using 6 cm petri dishes (surface area: 21 cm²) as follows:

1) Pour 10 mL of slurry into the 6 cm petri dish.

2) Turn on the lyophilizer and set the shelf temperature to −45 degrees Celsius. (Temperature controls pore size; the colder the initial temperature, the smaller the pores. −45 degrees Celsius gives average pore sizes between 70 μM+/−30 μM which we consider ideal for fibroblasts to migrate through and inhabit)

Note: the machine takes several hours to freeze the shelf at −45 C so flip it on as soon as you get to the lab, or you will have a long day/night.

3) Place samples onto the shelf and allow to freeze for 2 hours minimum.

4) Turn on the vacuum, and increase the temperature to −2 degrees C. and allow to lyophilize for 20-30 hours. Lyophilization is the process in which a solid (acetic acid and water in this case) is removed from a solution while AVOIDING the liquid phase, going from solid directly to gas. This is critical as if we simply added heat, the matrix would be desiccated and this would collapse the pores. Note: The time for this process varies with the amount of liquid to be removed. We set 20 hours for this step as we have observed incomplete lyophilization runs at 16 and 18 hours.

5) When ready to remove samples, increase shelf temperature to 20 C, approx. room temperature.

6) Once temperature has been achieved, turn the vacuum off and the lyophilizer off, remove samples. Note: Do not leave them in a warm area, they are fragile until crosslinked.

### EXAMPLE 8 (Addition of a Crosslinking Agent)

As mentioned above in Example 7, samples cannot be left in a warm area, and are fragile until crosslinked.

Crosslinkers are molecules to bond proteins together, thus creating a stronger matrix resistant to handling. Once added, the fibroblasts will remodel the matrix to their liking; without crosslinking the matrix would fall apart in several days. 1-ethyl-3-(-3-dimethylaminopropyl) carbodiimide (EDC) is a commonly used crosslinker for collagen, C6S, and even HA. In addition to EDC, there are other agents that act as chemical bonders for the matrix, such as: N-hydroxysuccinimide (NHS); glutaraldehyde; certain forms of gamma-irradiation and dehydrothermal crosslinking.

1) Create EDC solution: determine how much EDC solution will be needed. We use 1 mL EDC per 1 mL slurry+1 mL EDC. That means for a single 6 cm petri dish in which we added 10 mL of slurry, we would use 11 mL of EDC to ensure the matrix is completely covered. EDC has two reported concentrations, 20 mM and 50 mM. We have been using 20 mM.

2) 20 mM EDC recipe: 191.7 g of EDC per 1000 mL ethanol will be 1 M concentration. So, 3.834 g EDC per 1000 mL ethanol will be 20 mM concentration. Then scale down appropriately for the volume you desire to make.

3) Remove samples from lyophilizer and add solution of EDC to begin crosslinking allow to sit in solutions for 20 hours minimum.

Note: Ethanol will evaporate at warmer temperatures and this can collapse the matrix, so keep matrices at room temperature or colder area!4) Remove EDC, wash for 60 minutes with 50% ethanol/H₂O, followed by three additional washes (minimum) of distilled water for 15 minutes each.

Note: Approximately 50% of the HA can be lost during the wash steps; this can be corrected by starting the process with twice as much HA as you intend to have in your final volume. 5) Place samples back into the lyophilizer (tray at −45 C) and perform a second lyophilization to remove any residual water.

### EXAMPLE 9 (Matrix Creation Time)

For slurry production: about 4 hours time for 250 mL.

Lyophilization Time: approx. 72 hours total (initial 20-30 hr Lyophilization total EDC crosslinking and wash final Lyophilization)

The above described embodiments are set forth by way of example and are not limiting. It will be readily apparent that obvious modifications, derivations and variations can be made to the embodiments. The claims appended hereto should be read in their full scope including any such modifications, derivations and variations.

### EXAMPLE 10

Example 4, 5 or 6 is performed except for omitting step 3).

### EXAMPLE 11

Example 4, 5 or 6 is performed with a modified step 4 performed in a cold room.

### EXAMPLE 12

An above example is performed, using a water-soluble EDC.

### EXAMPLE 13

An above example is performed, with a shortened lyophilization time.

### EXAMPLE 14

An above example is performed, using a salt such as, e.g., sodium chloride, magnesium chloride, potassium chloride, sodium acetate, potassium hydroxide, sodium hydroxide, calcium acetate, sodium bicarbonate, etc. Such salt usage is for raising the ionic strength of the slurry, which aids in homogenization.

### EXAMPLE 15 (Vitamins)

In this example, a slurry is infused with at least one vitamin, such as vitamin A, vitamin C, vitamin D, combinations thereof, etc.

### EXAMPLE 16 (Further Examples of GAG Components)

Further examples of GAGs useable in the invention for matrix-production are chitosan, heparin sulfate, keratin sulfate, dermatan sulfate, and heparin.

### EXAMPLE 17

In this example, one or more GAG(s) is added (such as added to a slurry being formed) during matrix-production, and optionally one or more protein(s) is added, as follows: GAGs: C6S, HA, FN, chitosan, heparin sulfate, keratin sulfate, dermatan sulfate, heparin. Proteins: tenascin, elastin.

### EXAMPLE 18 (Product Matrices Lyophilized Using Metal Trays with Anodized Coating)

In this example, a metal tray comprising an anodized coating (such as a metal tray made to order by a metal-working shop) is used in the lyophilization step. Examples 18A-18E are some trays useable in a lyophilization step.

### EXAMPLE 18A

An aluminum tray comprising an anodized coating.

### EXAMPLE 18B

An aluminum tray comprising a chromate conversion coating.

### EXAMPLE 18C

A stainless steel tray comprising an anodized coating.

### EXAMPLE 18D

A metal tray comprising an anodized coating.

### EXAMPLE 18E

A tray comprising a chromate conversion coating.

### EXAMPLE 18F

An aluminum tray comprising 6061T1 chromate conversion coating as the anodized coating was used in the lyophilization step of a matrix production process. These aluminum trays comprising the 6061T1 chromate conversion coating are associated with a spectacular, unexpected result, of influencing capacity to manufacture matrices with more precise pore sizes.

The above described embodiments are set forth by way of example and are not limiting. It will be readily apparent that obvious modifications, derivations and variations can be made to the embodiments. Accordingly, the claims appended hereto should be read in their full scope including any such modifications, derivations and variations.

1-37. (canceled)

38. A method of making a matrix useable in a wound, comprising the steps of:
a) containing a slurry in a matrix carrier, which is not a tray, wherein the matrix carrier comprises a wound-shaped cavity having a size and shape duplicative of a wound;
b) performing a lyophilization step on the slurry while contained in the matrix carrier.

39. The method of claim 38, wherein in the containing step, the slurry comprises collagen and chondroitin sulfate.

40. The method of claim 39, wherein in the containing step, the slurry comprises collagen, chondroitin sulfate and HA.

41. The method of claim 40, wherein in the containing step, the slurry comprises collagen, chondroitin sulfate, HA and fibronectin.

42. The method of claim 38, wherein in the containing step, the slurry comprises acetic acid.

43. The method of claim 1, wherein the slurry-formulating step proceeds for a time in a range of about 30-120 minutes, at a temperature in a range of about 0° C. to 10° C.

44. The method of claim 1, wherein the lyophilizing step proceeds for a time in a range of about 24-72 hours, at a temperature in a range of about 0° C. to −80° C.

45. The method of claim 38, wherein the slurry-containing step proceeds for a time in a range of about 30-120 minutes , at a temperature in a range of about 0° C. to −80° C.

46. The method of claim 38, wherein the lyophilizing step proceeds for a time in a range of about 24-72 hours, at a temperature in a range of about 0° C. to −80° C.

47. The method of claim 1, wherein in the slurry-formulating step, a ratio of collagen to C6S is in a range of about 0.5% to 0.01%.

48. The method of claim 38, wherein after the aqueous components have been removed by the lyophilization step, a ratio of collagen to C6S is in a range of about 2% to 98%.

49. The method of claim 38, wherein the matrix carrier has no top cover and has a volume in a range of about 0.001 L to 50 L.

50. The method of claim 38, wherein the matrix carrier has a uniform height, and a height of the matrix carrier is in a range of about 0.01 inch to 10 inches.

51. The method of claim 38, wherein the matrix carrier has variable length dimensions and variable width dimensions, with a width dimension in a range of about 0.01 inch to 10 inches and a length dimension in a range of about 0.01 inch to 10 inches.

52. The method of claim 1, wherein in the lyophilization step, a tray is used wherein the tray is selected from the group consisting of a stainless steel tray, a stainless steel tray comprising an anodized coating, an aluminum tray, an aluminum tray comprising an anodized coating, and a tray comprising an anodized coating.

53. The method of claim 38, wherein in the lyophilization step, a tray comprising a chromate conversion coating is used.
